(19) United States
(12) Patent Application Publication
(10) Pub. No.: US 2005/0255111 A1
(43) Pub. Date: Nov. 17, 2005

(54) Use of an inhibitor or antagonist against tissue factor

(76) Inventors: Olle Korsgren, Uppsala (SE); Bo Nilsson, Uppsala (SE)

Correspondence Address: YOUNG & THOMPSON, 745 SOUTH 23RD STREET, 2ND FLOOR, ARLINGTON, VA, 22202, US

(21) Appl. No.: 10/505,279

(22) PCT Filed: Feb. 21, 2003
(86) PCT No.: PCT/SE03/00289

§ 371 Date: Jun. 6, 2005

(30) Foreign Application Priority Data
Feb. 22, 2002 (SE) 0200545-2
Nov. 28, 2002 (SE) 0203540-0

(51) Int. Cl. A61K 38/28; A61K 31/727; A61K 39/395

(52) U.S. Cl. 424/145.1; 514/3; 514/56

(57) ABSTRACT

The present invention relates to use of an inhibitor or antagonist against tissue factor, TF, in the production of a drug for treatment or prevention of diabetes or diabetes related diseases. The inhibitor or antagonist is mainly intended for treatment of diabetic patients suffering from type I or type II diabetes, respectively, as well as the metabolic syndrome preceding type II diabetes. The inhibitor or antagonist is an agent which completely or partially inhibits TF productions, such as an anti-TF antibody or an antisense construct acting on the TF gene.

### FIELD OF THE INVENTION

The present invention relates to use of an inhibitor or antagonist against tissue factor, TF, for production of a drug for treatment or prevention of diabetes or diabetes related diseases. The inhibitor or antagonist is mainly intended for treatment of diabetic patients suffering from type I or type II diabetes, respectively, as well as the metabolic syndrome preceding type II diabetes.

In the former case (type I diabetes) the antagonist or inhibitor is used in association with transplantation of islets of Langerhans to type I patients to enhance the survival of the islets. In the latter case the antagonist or inhibitor is used to prevent artheriosclerosis and cardiovascular disease seen in type II diabetic patients.

### BACKGROUND OF THE INVENTION

Haemostasis is crucial for survival. Any disturbance in the hemostatic balance such as damage to a vessel wall, leads to an immediate activation of the coagulation system. In vivo the coagulation system is mainly triggered by the 47-kDa transmembrane glycoprotein tissue factor (TF) which acts as a co-factor for the cleavage of factor VII to VIIa, for the proteolytic function of factor VIIa of the tissue factor (extrinsic) pathway of coagulation and as a receptor. TF belongs to the cytokine receptor superfamily and has, when complexed with factor VIIa, been shown to trigger intracellular signal transduction involved in angiogenesis, diapedesis, and inflammation. TF is constitutively expressed by cells in the adventitia of the blood vessels, and also in richly vascularised tissues such as the placenta, the brain and the lungs. Normally, cells exposed to blood such as endothelial cells and monocytes do not express TF, but certain inflammatory stimuli such as LPS, immune complexes and cytokines can induce TF expression in these cells. TF is strictly regulated by tissue factor pathway inhibitor (TFPI) In blood. Several recent publications have presented evidence of minute amounts of cryptic TF in blood that can be activated by unidentified stimuli. The blood borne TF is considered to allow an immediate activation of the coagulation cascade but is also a likely contributor to TF found in arteriosclerotic plaques. The origin of blood-borne TF is hitherto unknown.

There is a potential coupling between hyperinsulinemia/hyperglycemia and activation of the coagulation cascade. Ceriello et al reported that coagulation activation is increased after a meal. This was further underscored by studies showing that infusion of glucose induced a transient increase in the generation of FVIIa, reflecting TF pathway activation, and in thrombin generation in normal subjects. This effect was even more pronounced in patients with diabetes mellitus type 2 who had prolonged periods of hyperglycemia/hyperinsulinemia. Notably, the same level of hyperglycemia combined with simultaneous infusion of insulin that reduces the internal insulin secretion, abrogated the TF pathway activity. Not only individuals with diabetes mellitus type 2 but also those with high BMI, i.e. individuals with insulin resistance and hence increased production and levels of circulating insulin, have increased TF pathway activity. This hypercoaguble state in patients with diabetes mellitus type 2 is a conceivable explanation for the increased risk for diabetic vascular complications in this patient group.

Another potential association between TF and the islets of Langerhans is the clotting reaction triggered by islets exposed to ABO compatible blood both in clinical islet transplantation and in experimental studies. This reaction, designated instant blood-mediated inflammatory reaction (IBMIR), is characterised by an initial activation of the coagulation and complement systems, rapid binding and activation of platelets, binding of leukocytes, together resulting in disruption of islet integrity, and leading to a thrombus surrounding the islets.

For many years clinical islet transplantation had a success rate, assessed as insulin-independence after 1 year, of approximately 10%. Last year Shapiro et al. made a breakthrough in that they showed that insulin-independence could be obtained if the patient was treated with repeated transplants from more than one donor. In a follow-up study the same group showed that the transplanted patients had a β-cell function corresponding to only 20% of healthy individuals despite that the patients had received islets from more than one donor. Combined, these findings underscored that an adverse process, most likely loss of transplanted tissue, must be involved.

### SUMMARY OF THE INVENTION

The present inventors surprisingly found that tissue factor is expressed in human islets of Langerhans. Tissue factor was found in most of the endocrine cells within the islets but not in those of the exocrine tissue. The unexpected finding that TF is expressed and produced by the endocrine cells in the islets of Langerhans (“islet produced TF”) indicates that TF is released in association with release of insulin. This TF is most likely responsible for the increased risk of artheriosclerosis and cardio-vascular disease in patients with diabetes type II or pre-stages thereof and subjects with impaired glucose tolerance. Thus, an inhibitor or antagonist against TF production in the islets of Langerhans or release of tissue actor, or at least an active form thereof, from the islets, can be used to prevent artheriosclerosis and cardiovascular disease in these patients.

It is reasonable to believe that IBMIR explains the initial tissue loss that occurs during clinical islet transplantation. The trigger of IBMIR is not known but the present inventors have shown that EBMIR can be abrogated in vitro by Melagatran, a thrombin inhibitor, indicating that IBMIR is critically dependent on the activation of thrombin. Thrombin can be generated by two pathways: the tissue factor pathway (extrinsic pathway) and an amplification loop involving the intrinsic pathway. Hence, a local production of TF in human islets is most likely the initiator of IBMIR. Thus, an inhibitor or antagonist against TF can suppress or eliminate the IBMIR and this strategy can be used for treatment of type I diabetes patients in association with islet transplantation to enhance survival and avoid rejection of transplanted islets.

Thus, in a first aspect the invention relates to use of, or method of using, an inhibitor or antagonist against tissue factor, TF, in the production of a drug for treatment or prevention of diabetes or diabetes related diseases. The expression “diabetes or diabetes related diseases” includes impaired glucose tolerance or insulin resistance with unnormal production of insulin (e.g. hypersecretion of insulin) and diseases resulting from such metabolic conditions like arteriosclerosis, cardiovascular diseases (e.g. acute myocardial infarction) and cerebrovasclular diseases (e.g. bleeding and infarction)

The inhibitor/antagonist may be any agent that affects TF on the DNA, RNA or protein level, and can thus be selected from the group of known TF inhibitors, although the agent is not restricted to those.

In the context of the present invention, the expression “inhibition of TF” means complete or partial inhibition of TF production, as well as release of TF especially an active form thereof, from the islets. The invention further comprises the inhibition of released islet produced TF. By “inhibitor” we mean substances capable such inhibition of TF.

In a first embodiment the invention provides use as above in the production of a drug for administration in association with transplantation of insulin producing cells to patients with insulin dependent diabetes mellitus, IDDM.

In a second embodiment the invention provides use of an inhibitor/antagonist against TF for the production of a drug for treatment of cardiovascular diseases and/or artheriosclerosis. Free TF binds to artheriosclerotic plaques and contribute to the increased risk of thrombosis in, for example, myocardial infarction. This use is expected to be especially important for treatment of patients with type II diabetes or pre-stages thereof.

The inhibitor or antagonist against TF may be an anti-TF antibody having biological effect of binding TF especially islet produced TF.

The inhibitor or antagonist against TF may also be an agent capable of blocking the synthesis of TF, such as an antisense construct blocking the TF gene.

In an alternative embodiment, the inhibitor or antagonist against TF is used in combination with an anticoagulant such as heparin or fractions or derivatives thereof. Other possible combinations comprise a thrombin inhibitor and/or platelet inhibitor.

In a second aspect, the invention relates to a method of treatment or prevention of diabetes or diabetes related diseases, comprising administration of an inhibitor or antagonist against tissue factor, TF, to a subject in need thereof. The method is, for example, intended for treatment of diabetic patients and patients with impaired glucose tolerance. The method comprises administration of an anti-TF agent which completely or partially inhibits TF production in or release from the islets of Langerhans.

In a third aspect, the invention relates to inhibitors/antagonist per se which have the property of inhibiting, completely or partially, TF production in or release from the islets of Langerhans.

### DETAILED DESCRIPTION OF THE INVENTION

The invention will now be described in association with the accompanying drawings the contents of which are briefly described below:

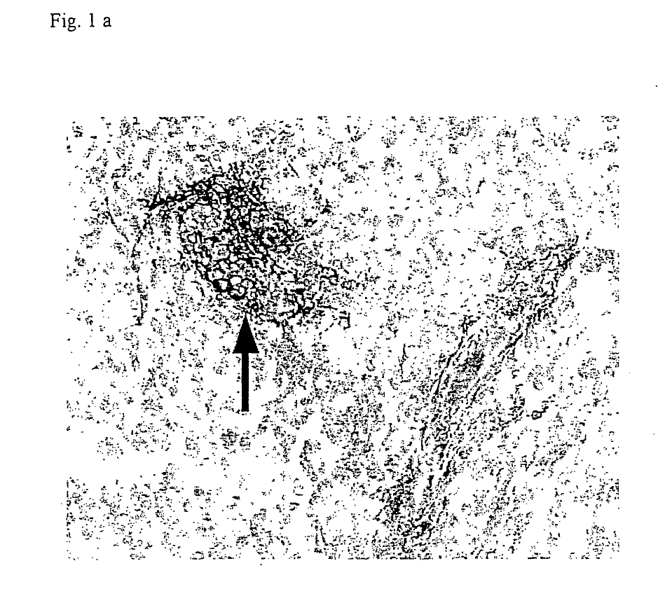

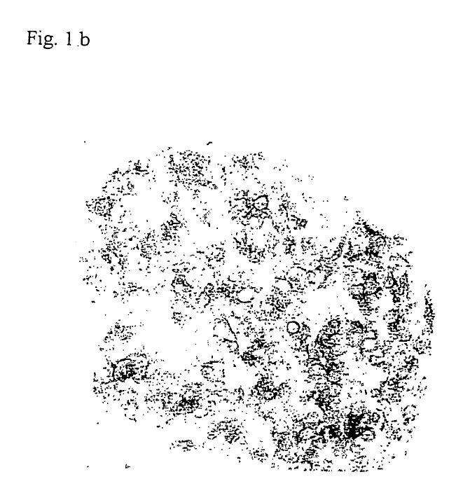

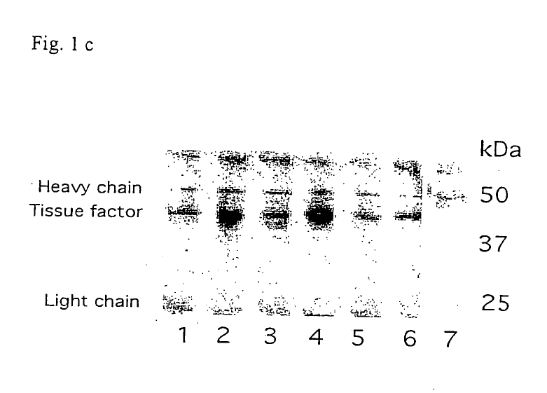

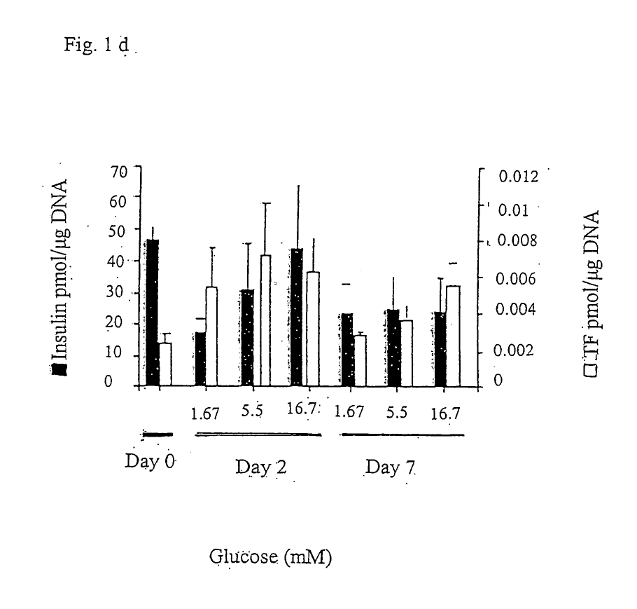

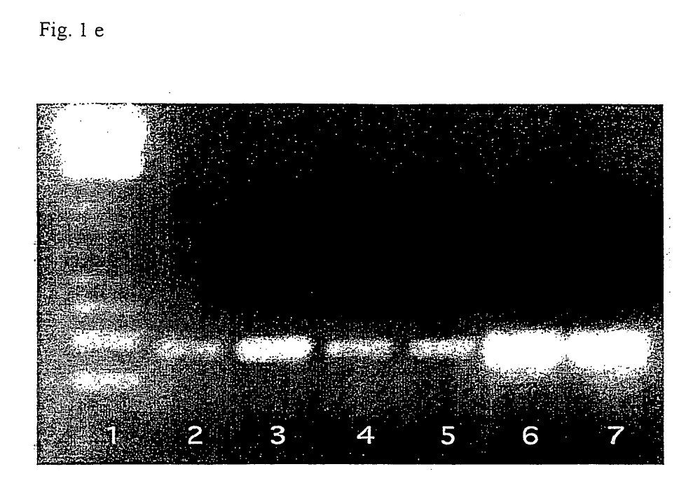

FIG. 1: Panel a. A section of human pancreas stained with mAb # 4509 shows a distinct staining of the pancreatic islets.
Panel b. A section of an isolated human islet stained with mAb anti-TF # 4509. TF is found in the majority of cells of the islet.
Panel c. Pure human islets from three individuals were immunoprecipitated with either mAb anti-TF # 4503 or 4509 and subjected to SDS-PAGE and Western blotting using rabbit polyclonal anti-TF #4502. Lanes 1-6 represent three pairs of TF precipitated with mAb 4503 (lanes 1, 3, 5) and mAb 4509 (lanes 2, 4, 6); lane 7 precipitating antibody alone.
Panel d. RT-PCR of isolated human islets from 6 individuals (lanes 2-7) yielding a 0.3 kb product. Lane 7 contains the molecular weight standard.
Panel e. Quantification of TF in human islets cultured for 0, 2 and 7 days (n=3).

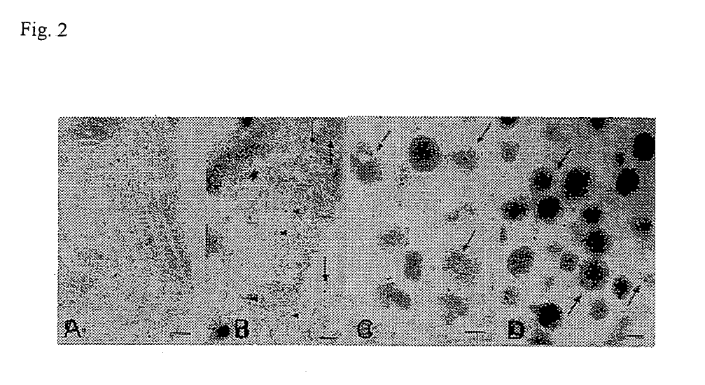

FIG. 2. Electron micrographs showing representative results from immuno-gold labelling with the mAb anti-TF # 4509 on sections from isolated islets. All goldparticles are 15 nm in diameter. All bars indicate 100 nm.
a) TF molecules (arrows) demonstrated in the smooth endoplasmic reticulum of a β-cell (x54000).
b) A Golgi apparatus in an α-cell with gold particles demonstrating TF molecules in the Golgi stacks (arrowheads), in transitory vesicles which are budded of from the Golgi trans region (large arrows) and in a secretory granule (small arrow) (x36000).
c) TF (arrows) storage in the core of the β-cell granules (x36000).
d) TF (arrows) storage randomly distributed in the α-cell granules (x 54000).

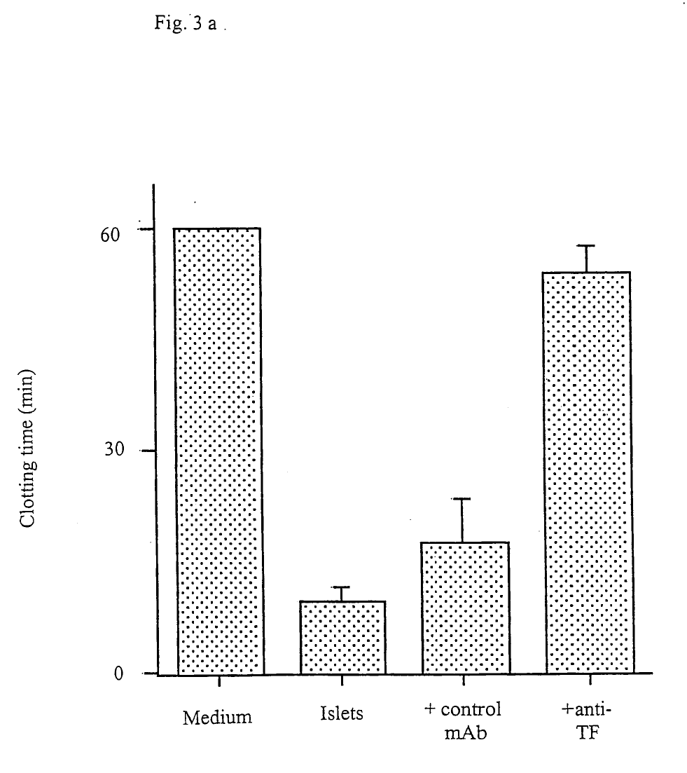

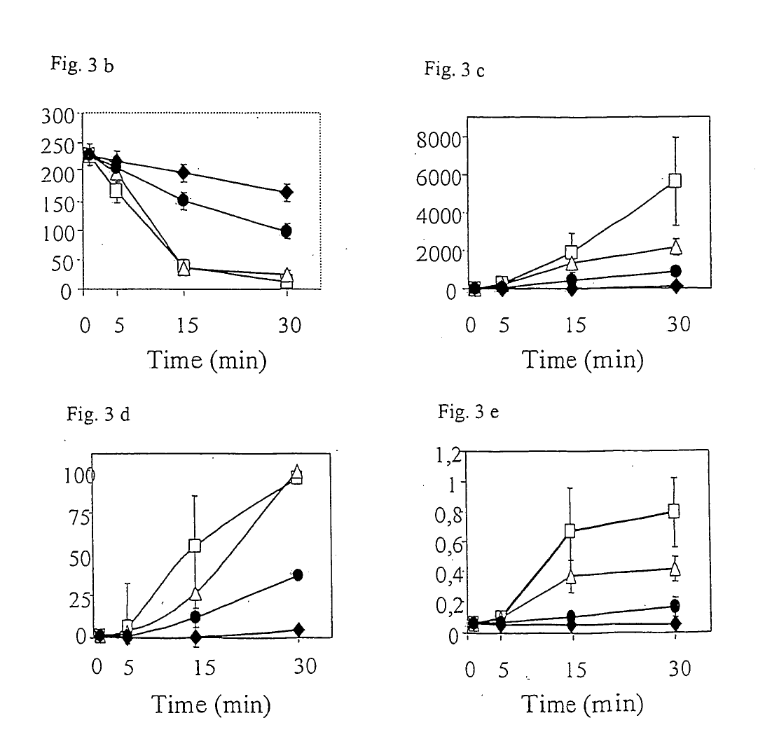

FIG. 3: Panel a: The effect of anti-TF on the clotting time triggered by human islets. Four μl of human islets were pretreated with medium, non-inhibitory mAb anti-TF #4503 (control mAb), and inhibitory anti-TF #4509 for 10 min at room temperature. Thereafter, the islets were incubated with 250 μl of non-anticoagulated human plasma and the clotting time was monitored in a ReoRox™ device (n=7). IBMIR induced by islets in the tubing loop model. The islets were pretreated with medium (open squares), non-inhibitory nab anti-TF #4503 (control mAb) (open triangles), and inhibitory anti-TF #4509 (filled circles) for 10 min at room temperature. Medium without islets is represented by filled diamonds. Thereafter, 4 μl of human islets were incubated with 5 ml of non-anticoagulated human ABO-compatible blood in heparinized tubing loops. IBMIR was monitored by b) platelet count, and EIAs for c) TAT, d) F 1+2, and e) FXIa-AT.

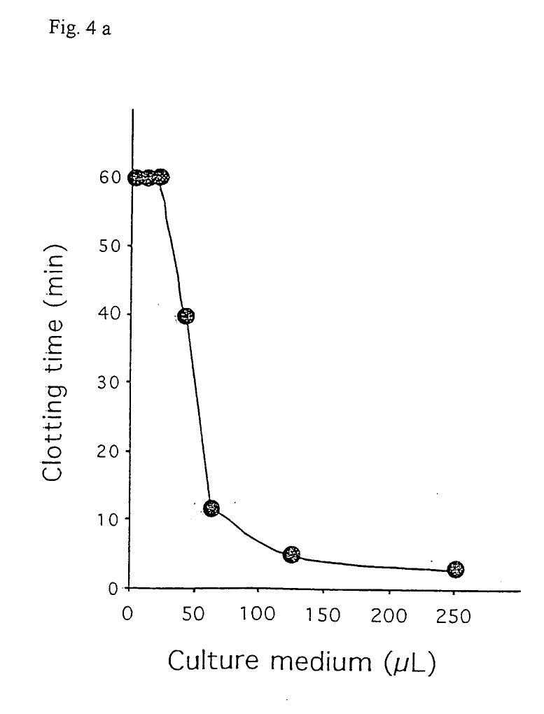

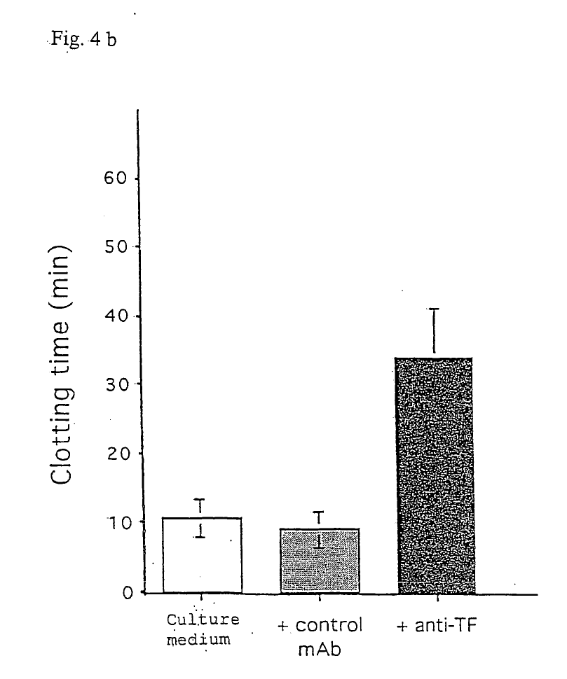

FIG. 4: Two hundred fifty μl of human citrate plasma was mixed with varying volumes of medium from cultures of human islets of Langerhans. The clotting time was assessed after recalcification of the plasma in a ReoRox™ device. Panel a is a representative serial dilution of medium from one islet batch while panel b is 60 μL of culture medium from islets of three different individuals which have been treated with PBS, mAb 4503 or mAb 4509 (mean±SEM).

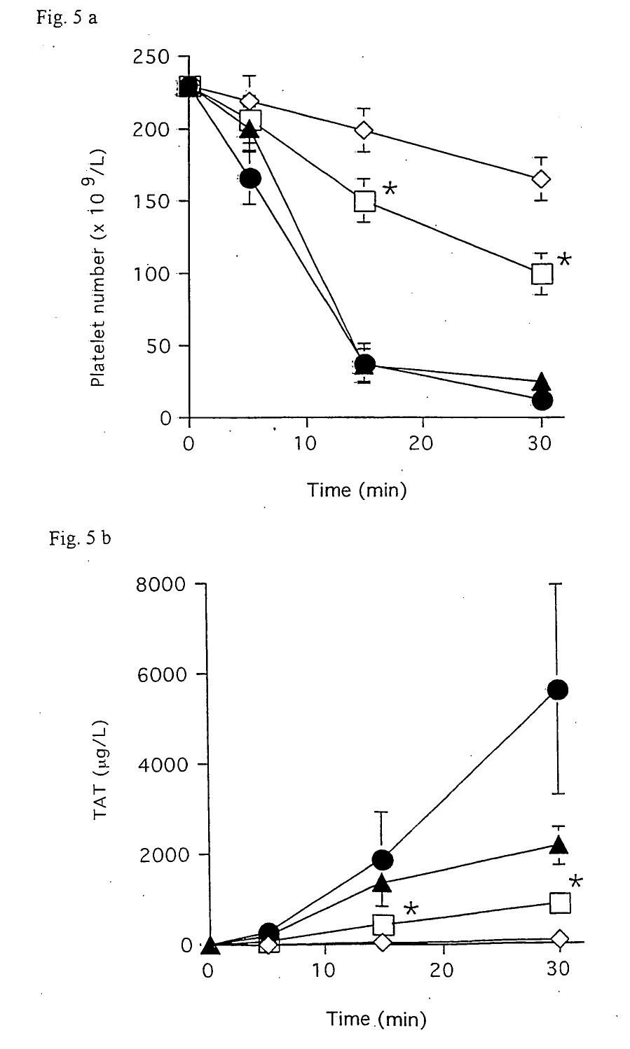

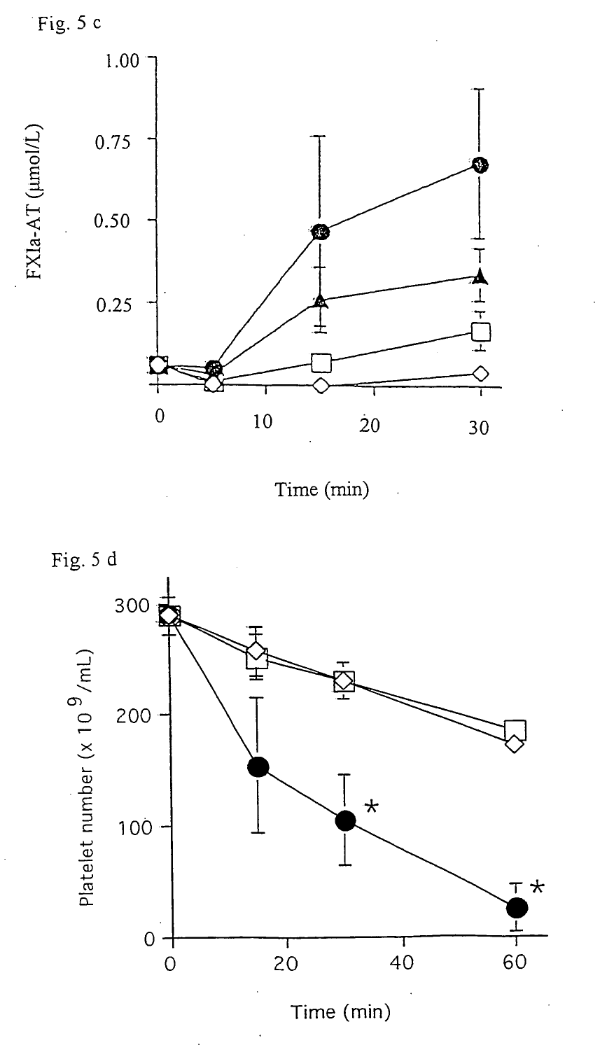

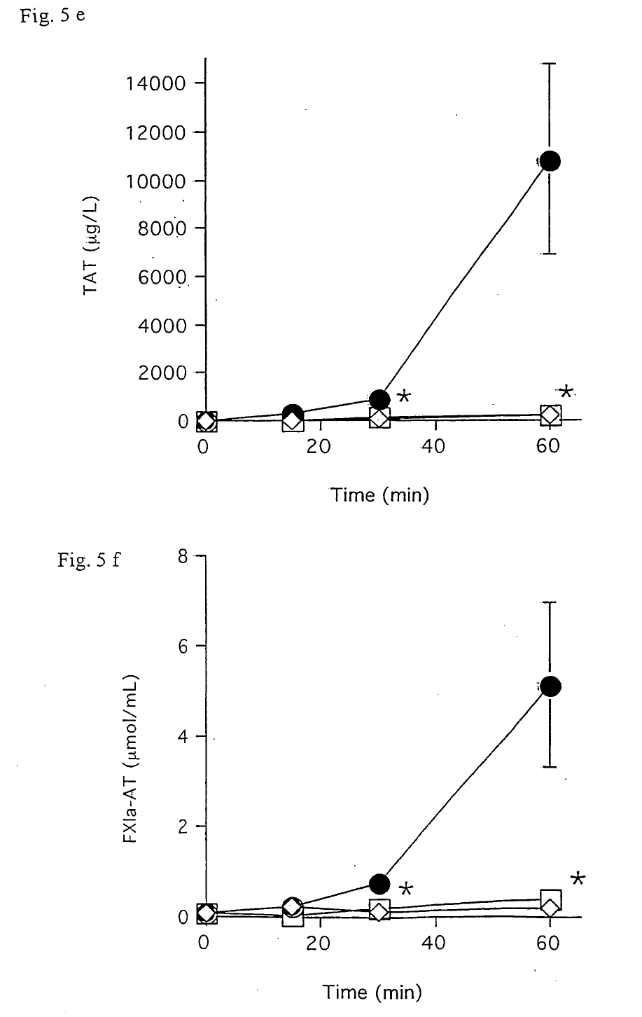

FIG. 5: IBMIR triggered by human islets is blocked by anti-TF and iFVIIa. IBMIR induced by islets in the tubing loop model. The islets were pretreated with PBS (●), non-inhibitory anti-TF mAb 4503 (control mAb; ▴), or inhibitory anti-TF mAb 4509 (□). ⋄ indicates medium without islets. The islets were incubated with non-anticoagulated human ABO-compatible blood in heparinised tubing loops. IBMIR was monitored by a) platelet count and ELAs for b) TAT, and c) FXIa-AT. (*p<0·05 when compared with the loop containing islets alone). Alternatively, the islet incubation was carried out in the presence (□) or in the absence (●) of 40 pmol/L of iFVIIa. ⋄ indicates medium without islets. IBMIR was monitored by d) platelet count and EIAs for e) TAT, and f) FXIa-AT. (*p<0.05 when compared with the loop without iFVIIa).

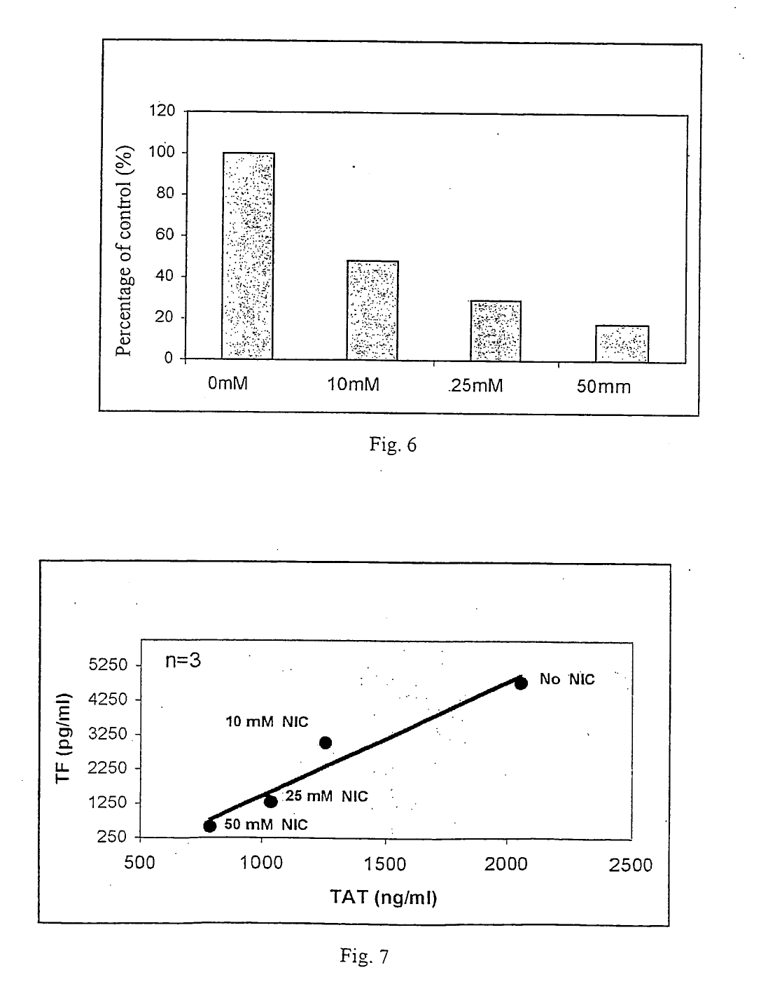

FIG. 6: The intracellular TF concentration after culturing in a medium containing nicotinamide, and assessed in comparison to a control.

FIG. 7: Generation of TAT, reflecting coagulation activiation.

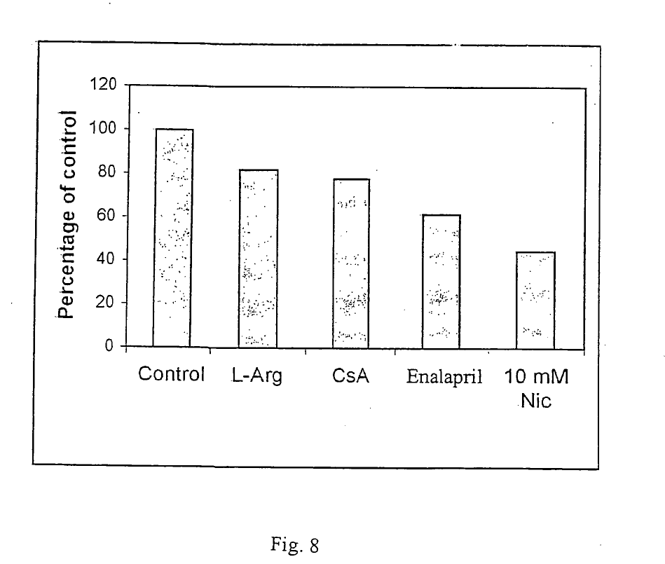

FIG. 8: The intracellular TF concentration after culturing in a medium containing Enalapril (40 μg/ml), Cyclosporin A (10 μmol/L), L-Aginine (1 mmol/L), Nicotinamide (10 mmol/L), respectively, and assessed in comparison to a control.

### DETAILED DESCRIPTION OF THE INVENTION

The Expression and Production of TF in Human Islets

Human pancreas sections were stained for the presence of TF with mAb 4509 (FIG. 1a). TF was found distributed in most, but not all, of the endocrine cells of the islets of Langerhans. The staining pattern suggested that TF was located in the granules of a majority of the islet cells. Also isolated islets from the human pancreas showed a similar distribution of TF showing that TF expression was not affected by the isolation procedure (FIG. 1b). TF was also found in the adventitia of larger blood vessels. The endothelial cells were not stained indicating that TF was not expressed in response to any inflammatory signals. No TF was found in the acinar cells of the exocrine pancreas.

In order to confirm that TF was present in human islets, the protein was pulled out from lysates of pure islets with two different mAb (#4503 and 4509) anti-TF antibodies. The antibody-bound protein was run on SDS-PAGE followed by Western blotting. The precipitated protein was identified using polyclonal anti-TF. In similar experiments TF was pulled out with polyclonal anti-TF and identified with either of the two mAbs (not shown). The polypeptide had a molecular weight of 47 kDa identical to that of TF (FIG. 1c). The amount of TF per cultured islet was calculated after quantification of TF in the lysates by ELISA (FIG. 1d). Directly after islet isolation the content was 13 pg/islet. It increased temporarily in culture, three times on day 2 (42 pg/islet) but was considerably lower on day 7 (21 pg/islet). RT-PCR performed on pure handpicked isolated islets confirmed the expression of TF also on the mRNA level (FIG. 1e).

Electron Microscopical Detection of TF in Human Islets

Electron microscopical demonstration of TF was performed on in situ islets from two normal pancreases and two batches of isolated islets using the immuno-gold technique on low temperature processed Lowicryl embedded specimens (FIG. 2). Endocrine cells in all examined islets were well preserved at the ultrastructural level although the contrast of intracellular structures was not optimal since the tissues were not treated with osmium. In both the in situ pancreatic islets and the isolated islets, gold particles demonstrated the presence of TF both in x- and β-cells. In both cell types, TF was localised in the smooth endoplasmic reticulum (FIG. 2a), in the Golgi stacks and in transitory vesicles budding of the trans-Golgi stacks (FIG. 2b) and also in the hormone granules of α- and β-cells (FIGS. 2c and d). The TF molecules were detected at a moderate concentration in the β-cell granules, preferably in the electron-dense core, but at a higher concentration and randomly distributed throughout the matrix of the α-cell granules. No TF immunoreactivity was shown in the δ- or PP-cells. All negative control labelling experiments were negative.

Blockade of IBMIR with Anti-TF Antibodies

Human islets of Langerhans in contact with non-anticoagulated human blood plasma induced gelation after approximately 9.5 min as monitored by viscosimetry compared to the control with only buffer which induced no clotting within 60 min, indicating that the islets were able to induce coagulation activation (FIG. 3a). In order to link the expression of TF of the islets with the procoagulant activity attempts were made to block the activity with anti-TF antibodies. In the presence of an inhibitory anti-TF (mAb 4509) the gelation was delayed by 5.6 times to 53 minutes, compared to 1.8 times for the control mAb against a non-functional epitope of TF (mAb 4503).

In order to investigate if TF triggered IBMIR, human islets were perifused with fresh human ABO-compatible blood in the tubing loop model for 30 min (FIG. 3). The islets were incubated with anti-TF antibodies for 10 min and then washed three times before they were added to the tubing loops. In the medium control and with the non-inhibitory anti-TF (4503), clotting occurred within 15 min, but with the inhibitory anti-TF (4509) clotting was inhibited over the whole observation period. This was reflected in the consumption of platelets (platelet count), in the release of platelet a-granule content (b-thromboglobulin), and in the generation of thrombin-antithrombin, prothrombin fragments 1+2 and factor XIa-antithrombin complexes which were all suppressed by 4509 but not by 4503 (FIG. 3; table I). These findings demonstrate that TF was the trigger of IBMIR.

Clotting of Human Blood Plasma by TF Released from Cultured Pancreatic Islets

A procoagulant activity was found in the culture medium of cultured islets, when the medium was mixed with human plasma (FIG. 4). In the presence of culture medium, the plasma clotted within 5 min. The clotting activity was blocked by mAb 4509 while mAb 4503 had no effect. If the supernatants were ultracentrifuged at x100.000 g no clotting was seen with the supernatants while the pellet had double activity compared with the unseparated culture medium. This showed that the TF activity was associated with a high molecular weight fraction and since TF is a membrane-bound protein with a transmembrane part, the protein was most likely associated with microparticles.

Blockade of IBMIR with Anti-TF Antibodies and iFVIIa

Incubation of human islets of Langerhans with fresh human plasma lacking additives induced gelation (as monitored by viscosimetry) after approximately 9.5 min, whereas the buffer alone induced no clotting after 60 min, indicating that the islets were able to induce coagulation activation (n=7; not shown). In order to link the expression of TF in the islets with the procoagulant activity, we attempted to block the gelation with anti-TF antibodies. In the presence of an inhibitory anti-TF (mAb 4509), gelation occurred 5-6 times slower (after 53 min), as compared to 1-8 times slower for the islets exposed to a control mAb against a non-functional epitope of TF (mAb 4503).

In order to investigate whether the observed TF triggers the IBMIR, we perifused human islets with fresh human ABO-compatible blood in the tubing loop model for 30 min (FIG. 5). The islets were incubated with inhibitory or non-inhibitory anti-TF mAb for 10 min and then washed three times before being added to the tubing loops. In the control samples (blood with islets alone or with the non-inhibitory anti-TF mAb 4503), clotting occurred within 15 min, but with the inhibitory anti-TF (mAb 4509), clotting was inhibited over the whole observation period. This difference was reflected in the consumption of platelets (platelet count), in the release of platelet α-granule content (β-thromboglobulin), and in the generation of TAT, F1+2 and FXIa-AT, which were all suppressed by inhibitory mAb 4509 but not by non-inhibitory mAb 4503 (FIG. 5a-c; Table I). An even more pronounced inhibition of IBMIR was obtained with iFVIIa, an efficient inhibitor of TF activity. Blood containing 40 pmol/L iFVIIa completely inhibited the drop in platelet count and the increase in TAT, FXIa-AT, and C3a (FIG. 5d-h). The effect of these two inhibitors strongly indicates that TF is the trigger of the IBMIR.

Anti-TF Antibodies and Site-Inactivated FVIIa

Antibodies against human tissue factor (mAbs 4509 and 4503 and polyclonal antibody 4502) were purchased from American Diagnostica Inc. (Greenwich, Conn.). MAb 4509 inhibits TF activity, and mAb 4503 recognises a nonfunctional epitope of TF. Polyclonal goat anti-mouse/10 and 15 nm Au (GAM-G10/15) and polyclonal goat anti-rabbit/10 and 15 nM Au (GAR-G 10/15) were purchased from Amersham International (Amersham, Bucks, England). Site-inactivated factor VIIa (iFVIIa) was obtained by inactivating FVIIa (NovoSeven, Novo Nordic, Denmark) with dansyl-Glu-Gly-Arg chloromethyl ketone according to Wildgoose et al.

Discussion

The results in the present study unanimously indicate that TF is produced and secreted by the αand β-cells of the islets of Langerhans. Immunoprecipitation, RT-PCR and the electron microscopy studies point to an expression of TF by both the α- and β-cells, but not by δ or PP cells. The TF activity found in culture supernatants indicates that TF is released from the islets. The localization of TF to the α- and β-cell granules indicates that TF is released together with insulin and glucagon. Regulation of TF synthesis is, however, unknown.

The fact that IBMIR was inhibited by anti-TF combined with the recent finding that most of the process of IBMIR is driven and amplified by thrombin, allows us to propose a hypothesis of how this reaction is pushed forward, although we in no way regard us to be bound by this hypothesis, and the hypothesis must not be construed as narrowing on the scope of the invention as defined in the claims. After the initial generation of thrombin by islets-expressed TF, thrombin-activated platelets start to bind to the islet surface. The ligand/s to which the platelets bind to on the islet surfaces is still not identified but collagen types I, III, IV, and V have been reported to surround human islets. Collagen is a known mediator of platelet binding and activation. This is followed by a rapid loss of platelets from the blood. Via the amplification loop involving factor XI and activated platelets, more thrombin is formed which generates a fibrin capsule surrounding the islets.

Inhibition of islets-bound TF activity before transplantation is likely to prevent IBMIR in clinical islet transplantation. It is envisaged that pretreatment protocols consisting of agents able to block both TF expression (anti-TF antibodies) and agents capable of blocking TF synthesis e.g. anti-sense, will be developed in the near future. Pretreatment of the islets prior to transplantation would have clear clinical benefits since it would have no adverse effect on heamostasis of the recipient.

A potential source of blood-borne TF is leukocytes which are known to produce microparticle-bound TF. The findings in normal individuals of increased TF pathway activity in response to glucose infusion strongly indicate that TF-activity is initiated by an alternative glucose sensitive mechanism. The release of TF together with insulin would give such a close relationship in individuals with no apparent ongoing inflammatory process. Ruptured arteriosclerotic plaques contains TF and the amount of TF correlates with the thrombogenicity of the plaque. Since the concentration of TF is increased close to the luminal surface of the plaque, it has been proposed that the plaque-associated TF is derived from the blood. In support of this finding, blood-borne TF bind to arteriosclerotic plaques which subsequently is able to initiate the development of a local arterial thrombosis. The present study suggests that at least a fraction of the blood-borne TF originate from the pancreatic islet cells. Considering the increased generation of thrombin in response to hyperglycemia in type 2 diabetic patients and, possibly in other individuals with insulin resistance and hyperinsulinemia, more active TF is likely to be present and able to bind to arteriosclerotic plaques. This would increase the risk for thrombosis in patients with these conditions.

According to a further aspect of the invention, TF production and/or release thereof can be inhibited by the administration of insulin or other substances which will reduce insulin production.

It has been discovered that patients that have been given insulin will exhibit a significantly lowered rate of cardiovascular disease. A proposed explanation is that because TF is closely associated with insulin in the islets of Langerhan, where the insulin is produced, the rate of release of TF from the islets will be reduced concomitantly with the reduction of insulin release.

A proposed mechanism involved is that insulin will reduce the level of glucose in the blood, and the glucose sensing system will note this reduced glucose level, and hence will not trigger further release of insulin from the islets, thereby also reducing the release of TF.

At present patients suffering from diabetes type 2 are not treated with insulin. Instead, they are given medicaments triggering the release of insulin from the islets, with the accompanying release of TF, increasing the risk of CHD to occur.

This means that insulin can be used for the prevention of CHD in patients subject to a risk of being struck by CHD, e.g patient having diabetes type 2 and its prodromal condition, insulin resistance. Accordingly, the invention provides a new method for treatment of these patients comprising administration of a substance which can be characterized by reducing production and/or release of insulin, but act via TF inhibition.

Still another important finding within the scope of the invention is that there is a link between hyperinsulinemia and cardiovascular disease in patients suffering from type 2 diabetes. Thus, the discovery of a link between TF expression in the islets of Langerhan and the well known increased risk of coronary heart disease (CHD) in patients with type 2 diabetes, can be used within the scope of the present invention, for the purpose of providing medicaments and treatments.

Hyperinsulinemia is a feature that is common to both type 2 diabetes and its prodromal condition, insulin resistance. In both conditions, the β-cells of the pancreatic islets produce increasing amounts of insulin to control a relative hyperglycemia that is the result of a progressively increasing insulin resistance. Both these conditions are associated with an increased risk of CHD. Thereby, the risk of myocardial infarction in otherwise healthy type 2 diabetes patients is as high as that in patients who have already suffered an infarction.

It has been reported in several publications that coagulation activation occurs after a meal. These findings have been further supported by reports that infusion of glucose induces a transient increase in the generation of FVIIa, reflecting TE pathway activation, and in thrombin generation in normal subjects. The effect is even more pronounced in patients with type 2 diabetes mellitus, who experience prolonged periods of hyperinsulinemia/hyper-glycemia. Of particular interest is the observation that the same level of hyperglycemia, when combined with simultaneous infusion of insulin to reduce the endogenous insulin secretion, is able to abrogate the TF pathway activity, indicating that the endogenous production of insulin is a prerequisite for the coagulation activation.

The binding of microparticle-bound TF to platelets is thought to be of importance for the progression of a thrombus. In particular, blood-borne TF is significantly increased in patients with acute myocardial infarction and unstable angina.

The local production of TF in human islets and excretion in response to prolonged periods of hyperglycemia provide a tentative explanation for the activation of the systemic TF pathway during hyperinsulinemia.

Arteriosclerotic plaques contain TF, and the amount of TF correlates with the thrombogenicity of the plaque. The origin of TF in these plaques is not fully understood, but both smooth muscle cells and foam cells in the lipid core of the plaque are known to produce TF. The arteriosclerotic plaque can trigger thrombus formation in two ways: either the plaque ruptures and exposes its TF-containing lipid core, or the endothelial surface of the plaque is denuded, and the underlying tissue induces thrombus formation. In the latter case, blood-borne TF could adhere to the subendothelial surface and trigger thrombosis.

Therefore, in accordance with one aspect of the present invention the list of TF inhibitors includes substances that are capable of reducing insulin secretion and accordingly would lower the release of TF from the islets of Langerhans. The examples of such substances include thiazolinediones, which reduce insulin resistance, and/or exogenous insulin, or insulin analogues, native or recombinant.

Materials & Methods

Islet Isolation

We isolated islets as described elsewhere from human cadaver donors (approved by the ethical commitee), using a Liberase perfusion followed by a continous density ficoll gradient purification in a refrigerated COBE 2991 centrifuge (COBE Blood Component Technology, Lakewood, Colo., USA). The islet preparations were maintained in culture medium (CMRL 1066; ICN Biomedicals, Costa Mesa, Calif.) at 37° C. (5% CO₂) for 1-7 days. The volume and purity were determined by microscopic sizing after staining with diphenylthiocarbazone. Viability was assessed as insulin secretion in response to a glucose challenge in a dynamic perifusion system (in 1.67, 16.7 and back to 1.67 μmol/L glucose).

Anti-TF Anitibodies

Antibodies against human tissue factor (mAb # 4509, # 4503 and polyAb #4502) were purchased from American Diagnostica Inc. (Greenwich, Conn., USA)

Immunohistochemical Staining

Pieces of whole pancreases and isolated islets were collected in an embedding medium (Tissue-Tek; Miles, Eckhart, Ind.) and snap-frozen in liquid nitrogen. The samples were sectioned and stained with mAb # 4509 followed by HRP-conjugated swine anti-mouse Ig (DAKO A/S, Glostrup, Denmark).

Immunoprecipitation of TF from Purified Human Islets

Two thousand islets were washed five times by centrifugation at 9×g at room temperature (RT) with PBS containing 5 mM EDTA, 10 mM benzamidine, 0.1 mg/ml of soy bean trypsin inhibitor, and 1 mM PMSF. The pellet was incubated in 0.5 ml of the same buffer supplemented with 1% Triton X-100 (Sigma), at 37° C. for 30 min. Thereafter, the cell debris was removed by centrifugation at 10 000×g for 5 min. Three μg of mAb # 4509 or #4503 was incubated with 250 μl of the cell lysate for 30 min at 37° C. and precipitated with Protein G Sepharose (Pharmacia Upjohn, Stockholm, Sweden). The samples were subjected to SDS-PAGE and Western blot analysis using rabbit using polyAb #4502 and HRP-conjugated antibodies against rabbit immunoglobulins (Dako A/S).

Election Microscopy

For ultrastructural analysis, normal pancreatic tissue specimens from two male patients and isolated islets from two pancreas-donors were sampled. None of the patients or the donors suffered from any metabolic disease, all pancreases were macro- and microscopically normal and did not show any amyloid depositions. In order to preserve the antigenicity, the specimens were processed with the low temperature method. Ultrathin sections placed on nickel grids were immunolabelled with the immuno-gold technique. TF antibodies mAb# 4509, # 4503 and pAb # 4502, dilution 1:25)(see also Table 1), and 10 or 15 nm colloidal gold particles were used as electron-dense markers. Sections were contrasted with uranyl acetate and lead citrate before examination in a Philips 201 electron microscope.

Clotting Time

Clotting time in plasma was measured in a four channel free oscillating rheometer, ReoRox 4, from GHI (Global Haemostasis Institute AB, Linköping, Sweden).

Tubing Loops as a Model

We use a modification of a model previously described. Loops made of PVC tubing (diameter=6.3, length 390 mm) were furnished with the Corline heparin surface (Corline, Uppsala, Sweden) according to the manufacturer's recommendation.

Four μL (˜4,000 IEQ) of washed islets (twice in CMRL 1066) were either pre-incubated with 15 μl of mAb #4509, control mAb #4503 or with PBS for ten minutes at room temperature. After three washing steps the islets were resuspended in 150 μL of CMRL 1066 and placed in the loops, thereafter fresh ABO-compatible human blood (5 mL) was added. To generate a blood flow of approximately 100 mL/min we put them on a rocking device, placed in an 37° C. incubator for 30 minutes. We also included a control loop containing blood supplemented with 150 μL CMRL 1066 but no islets. Before perfusion and at 5, 15 and 30 minutes we collected blood samples in EDTA (4.1 mM, final concentration) for further analysis.

Blood and Plasma Analysis

Platelets and differential leukocytes in blood were counted using a Coulter AcT diff analyzer (Beckman Coulter, Fla., USA).

Plasma levels of Prothrombin fragment 1+2 (F1+2) and Thrombin-antithrombin complex (TAT) were quantified using commercially available EIA-kits (Enzygnost® F1+2 and TAT, Dade Behring, Marburg, Germany). Plasma FXIa-AT complex was quantified according to Sanchez et al. β-thromboglobulin (β-TG) was analyzed using Asserachom (Diagnostica Stago, Asnières-sur-Seine, France). Complement activation products C3a and sC5b-9 were determined as previously described.

RT-PCR Analysis

Cytoplasmic RNA from islets were isolated as described in Single stranded cDNAs were prepared using oligo(dT) priming (Amersham Pharmacia). PCR primers were combined to generate PCR products spanning two or more exons of the TF transcript to amplify cDNA only and not trace amounts of genomic DNA. RT-PCRs were conducted for 35 cycles by using high-fidelity PCR components (Expand, Boehringer-Mannheim, Germany), thereafter the products were analyzed on 3% agarose gels with 0.5 μg/ml of ethidium bromide.

Statistical Analysis

All results were expressed as mean±SEM. Mean values are compared using Friedman ANOVA. The significance was determined at α=0.05.

TABLE 1

| Antibodies used | Origin |
|---|---|
| 1. Monoclonal anti-human tissue factor antibody (#4509) | Mouse |
| 2. Polyclonal anti-human tissue factor antibody (#4502) | Rabbit |
| 3. Monoclonal anti-human tissue factor antibody (#4503) | Mouse |
| 4. Monoclonal anti-human insulin (XX) | Mouse |
| 5. Polyclonal goat anti-mouse/10 nm Au (GAM-G10) | Goat |
| 6. Polyclonal goat anti-rabbit/15 nm Au (GAR-G15) | Goat |

1, 2, 3 and 4: American Diagnostica Inc., Greenwich, CT, USA
5 and 6: Amersham International, Amersham, Bucks, England

Inhibition of TF Synthesis and Section

Islets were cultured for 24 h in CMRL-medium containing 10 nM Nicotinamide, which is the standard medium used for islet culture. The medium was thereafter exchanged to CMRL-medium without Nicotinamid. After a baseline period of another 24 h, islets were handpicked for analysis of TF content. Culture was continued for 48 h with medium containing agents known to affect TF expression in monocytes and endothelial cells. These were, L-Arginin, Cyclosporin A, Enalapril, acetylcysteine and nicotinamid. The islets were harvested, the TF content analysed using a commercial EIA kit and the islets tested in the in vitro loop model.

Culture of the islets together with the vitamin B nicotinamide reduced the synthesis and secretion of tissue factor. When the islets were cultured together with nicotinamide at concentrations ranging from 0 to 50 mM the production of tissue factor was dose-dependently inhibited in the islets (FIG. 1A). Similarly, when tested in the in vitro loop system in contact with human blood, the intracellularly content of TF correlated with the coagulation activation as reflected by the generation of TAT (FIG. 1B). This demonstrates that the TF content of the islet cells and the secretion of TF were inhibited by nicotinamide in a dose dependent manner. Similar effects were obtained with immunosuppressive drug cyclosporine A (FIG. 2), the amino acid L-Arg (FIG. 2), the angiotensine converting enzyme inhibitor Enalapril (FIG. 2), and the antioxidant acetylcysteine (not shown).

FIGS. 6-8 illustrate the effect of nicotinamide.

Islets were cultured for 48 h in medium containing 0, 10, 25 and 50 mM Nicotinamid and the intracellular TF concentration assessed, see FIG. 6. The islets were also exposed to fresh human ABO compatible blood in the in vitro loop model. Samples were retrieved after 5, 15, 30 and 60 min and analysed for TAT, see FIG. 7.

Islets were cultured for 48 h in medium containing Enalapril (40 μg/ml), Cyclosporin A (10 μmol/L), L-Aginine (1 mmol/L), Nicotinamide (10 mmol/L) and the intracellular TF concentration assessed. The concentration of Tf is expressed as percentage of the untreated control, see FIG. 8.

It is also to be noted that nicotinamide has anti-oxidative properties, which is believed to be an important factor in the mechanism responsible for its activity. Thus, it is anticipated that other compounds within this group of substance are usable in accordance with the invention. Further examples of such compounds, the enumeration being non-exhaustive, are vitamin E, glutathione, acetylcysteine, pyrrolidine dithiocarbamate, pyrithione, pentoxifylline, Hemoxygenase-1/CO-bilirubin, Prostaglanding A1.

1. A method for producing a drug for the treatment or prevention of diabetes or diabetes related diseases, comprising adding an inhibitor or antagonist against tissue factor (TF) to a pharmaceutically acceptable carrier.

2. A method for facilitating the transplantation of insulin producing cells to patients with insulin dependent diabetes mellitus, IDDM, comprising administering an effective amount of an inhibitor or antagonist against tissue factor (TF) to a patient in need thereof.

3. A method for the treatment of cardiovascular diseases and/or artheriosclerosis, comprising administering an effective amount of an inhibitor or antagonist against tissue factor (TF) to a patient in need thereof.

4. A method according to claim 3, said patient has type II diabetes or pre-stages thereof.

5. The method according to claim 3, wherein the inhibitor or antagonist against TF is an anti-TF antibody having biological effect of binding TF.

6. The method according to claim 1, wherein the inhibitor is a substance inhibiting production and/or release of TF from the islets of Langerhan.

7. The method according to claim 1, wherein the inhibitor or antagonist against TF is an agent capable of blocking the synthesis of TF.

8. The method according to claim 7, wherein the agent is an antisense construct blocking the TF gene.

9. The method according to claim 1, wherein the substance inhibiting production and/or release of TF is characterized by decreasing the production of insulin in the islets of Langerhan.

10. The method according to claim 9, wherein the substance is insulin or insulin analogue.

11. The method according to claim 3, wherein the inhibitor or antagonist against TF is administered in combination with an anticoagulant.

12. The method according to claim 3, wherein the inhibitor or antagonist against TF is administered in combination with a thrombin inhibitor.

13. The method according to claim 3, wherein the inhibitor or antagonist against TF is administered in combination with a platelet inhibitor.

14. A method of treatment or prevention of diabetes or diabetes related diseases, comprising administration of an inhibitor or antagonist against tissue factor, TF, to a subject in need thereof.

15. The method according to claim 14, wherein the inhibitor or antagonist against TF is administered in combination with an anticoagulant.

16. The method according to claim 14, wherein the inhibitor or antagonist against TF is administered in combination with a thrombin inhibitor.

17. The method according to claim 14, wherein the inhibitor or antagonist against TF is administered in combination with a platelet inhibitor.

18. The method according to claim 14, wherein the inhibitor is a substance inhibiting production and/or release of TF from the islets of Langerhan.
